(19) United States
(12) Patent Application Publication
(10) Pub. No.: US 2021/0345905 A1
(43) Pub. Date: Nov. 11, 2021

(54) PHYSIOLOGICAL SENSING SYSTEM

(71) Applicant: Active Insight Corporation, New Taipei City (TW)

(72) Inventors: Mao Chen Liu, Nantou County (TW); Jung-Ching Hsu, New Taipei City (TW)

(73) Assignee: Active Insight Corporation, New Taipei City (TW)

(21) Appl. No.: 16/894,890

(22) Filed: Jun. 8, 2020

(30) Foreign Application Priority Data
May 5, 2020 (TW) 109114904

(51) Int. Cl. A61B 5/08; A61B 5/00; A61B 5/01

(52) U.S. Cl. CPC A61B 5/08; A61B 5/746; A61B 5/01; A61B 2560/0204; A61B 5/7203; A61B 5/6801; A61B 5/7225

(57) ABSTRACT

A physiological sensing system is provided. The physiological sensing system includes a wearable device and an electronic device. The wearable device senses a body temperature signal of a user, an audio signal of a respiratory tract of the user, a capacitance sensing signal of the respiratory tract and an activity status of the user to generate a sampled body temperature signal, a sampled audio signal, a sampled sensing capacitance value and a sampled activity status. The electronic device determines a physiological status of the user in response to variations in the sampled body temperature signal, the sampled audio signal, the sampled sensing capacitance value and the sampled activity status.

### CROSS-REFERENCE TO RELATED APPLICATION

This application claims the priority benefit of Taiwan application no. 109114904, filed on May 5, 2020. The entirety of the above-mentioned patent application is hereby incorporated by reference herein and made a part of this specification.

### TECHNICAL FIELD

The invention relates to a physiological sensing system, and more particularly, to a physiological sensing system for sensing user's respiratory tract status.

### BACKGROUND

According to current technology, methods for determining whether the respiratory tract is infected by a virus mainly include collecting secretions from the larynx or nasal cavity to perform nucleic acid tests on the secretions, or performing chest X-ray examination. However, the above methods cannot perform a long-term monitoring on the respiratory tract status. In addition, collecting secretions from the larynx or nasal cavity and performing chest X-ray examinations are less convenient for the subjects. Therefore, how to realize the long-term monitoring on the respiratory tract status and provide a convenient detection method is one of the issues to be addressed by those skilled in the art.

### SUMMARY

The invention provides a physiological sensing system that can realize the long-term monitoring on the respiratory tract status and improve the convenience of detection.

The physiological sensing system of the invention includes a wearable device and an electronic device. The wearable device includes a sensing module and a processor. The sensing module is configured to sense a body temperature signal of a user, sense an audio signal of a respiratory tract of the user, sense a capacitance sensing signal of the respiratory tract, and sense an activity status of the user. The processor is coupled to the sensing module. The processor is configured to sample the body temperature signal, the audio signal and the activity status to generate a sampled body temperature signal, a sampled audio signal and a sampled activity status. The processor generates a sampled sensing capacitance value according to the capacitance sensing signal. The electronic device is configured to communicate with the wearable device to receive the sampled body temperature signal, the sampled audio signal, the sampled sensing capacitance value and the sampled activity status. The electronic device determines a physiological status of the user in response to variations in the sampled body temperature signal, the sampled audio signal, the sampled sensing capacitance value and the sampled activity status.

Based on the above, the invention can sense the body temperature signal of the user, the audio signal of the respiratory tract, the capacitance sensing signal of the respiratory tract and the activity status of the user to generate the sampled body temperature signal, the sampled audio signal, the sampled sensing capacitance value and the sampled activity status. The invention can determine the physiological status of the user in response to variations in the sampled body temperature signal, the sampled audio signal, the sampled sensing capacitance value and the sampled activity status. In this way, the invention can realize the long-term monitoring on the respiratory tract status and improve the convenience of detection.

To make the aforementioned more comprehensible, several embodiments accompanied with drawings are described in detail as follows.

### BRIEF DESCRIPTION OF THE DRAWINGS

FIG. 1 is a schematic diagram of a physiological sensing system illustrated according to an embodiment of the invention.

FIG. 2 is an equivalent circuit diagram formed by a respiratory tract and capacitance detecting electrodes illustrated according to an embodiment of the invention.

FIG. 3 is a schematic diagram of a wearable device illustrated according to an embodiment of the invention.

FIG. 4 is a schematic diagram of a wearable device illustrated according to another embodiment of the invention.

FIG. 5 is a schematic diagram of a usage scenario of the wearable device illustrated according to an embodiment of the invention.

### DETAILED DESCRIPTION

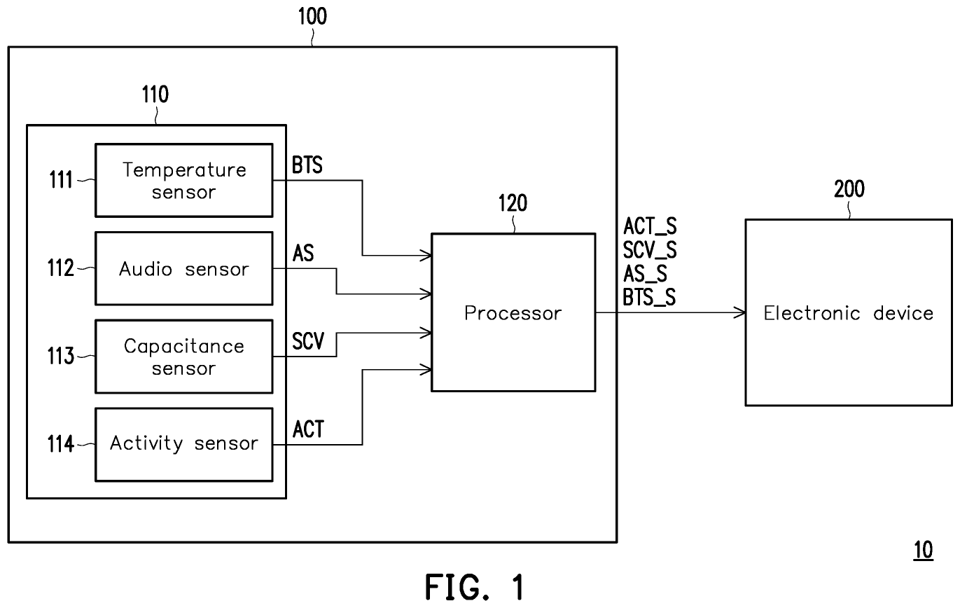

Referring to FIG. 1, FIG. 1 is a schematic diagram of a physiological sensing system illustrated according to a first embodiment of the invention. In this embodiment, a physiological sensing system 10 includes a wearable device 100 and an electronic device 200. The wearable device 100 includes a sensing module 110 and a processor 120. The wearable device 100 may be attached to the chest of a user so that the sensing module 110 can sense multiple physiological statues of the user. In this embodiment, the sensing module 110 can sense a body temperature signal BTS of the user, sense an audio signal AS of a respiratory tract of the user, sense a capacitance sensing signal SCV of the respiratory tract, and sense an activity status ACT of the user. In this embodiment, the sensing module 100 includes a temperature sensor 111, an audio sensor 112, a capacitance sensor 113 and an activity sensor 114. The temperature sensor 111 continuously senses the body temperature signal BTS of the user. The temperature sensor 111 may be implemented by, for example, an electronic thermometer. The audio sensor 112 continuously senses the audio signal AS of the respiratory tract. The audio sensor 112 may be implemented by, for example, a microphone. The capacitance sensor 113 continuously senses the capacitance sensing signal SCV of the respiratory tract. The activity sensor 114 continuously senses the activity status ACT of the user. The activity sensor 114 may be implemented by, for example, an accelerometer (G-sensor).

In this embodiment, the processor 120 is coupled to the sensing module 110. In this embodiment, the processor 120 is coupled to the temperature sensor 111, the audio senor 112, the capacitance sensor 113 and the activity sensor 114. Accordingly, the processor 120 can continuously receive the body temperature signal BTS, the audio signal AS, the capacitance sensing signal SCV and the activity status ACT. The processor 120 samples the body temperature signal BTS to generate a sampled body temperature signal BTS_S. The processor 120 samples the audio signal AS to generate a sampled audio signal AS_S. The processor 120 can filter the sampled audio signal AS_S to filter out signals outside a preset frequency range. In addition, the processor 120 can also gain the sampled audio signal AS_S, so that an intensity of the sampled audio signal AS_S may also be amplified. Accordingly, the sampled audio signal AS_S can meet the interpretable specific specifications. In this way, the sampled audio signal AS_S is easier to identify. The processor 120 can generate a sampled sensing capacitance value SCV_S according to the capacitance sensing signal SCV. For instance, the processor 120 can filter the capacitance sensing signal SCV to filter out noises accompanying the capacitance sensing signal SCV, and calculate the sampled sensing capacitance value SCV_S according to the capacitance sensing signal SCV. The sampled sensing capacitance value SCV_S corresponds to an equivalent capacitance value generated by an alveolar status in the respiratory tract. Further, the processor 120 can also sample the activity status ACT to generate a sampled activity status ACT_S.

The processor 120 of this embodiment is, for example, a central processing unit (CPU) or other programmable devices for general purpose or special purpose such as a microprocessor and a digital signal processor (DSP), a programmable controller, an application specific integrated circuit (ASIC), a programmable logic device (PLD) or other similar devices or a combination of above-mentioned devices, which can load in computer programs for execution.

In this embodiment, the electronic device 200 conducts a wired communication or a wireless communication with the wearable device 100 to receive the sampled body temperature signal BTS_S, the sampled audio signal AS_S, the sampled sensing capacitance value SCV_S and the sampled activity status ACT_S. For instance, the wearable device 200 may communicate with the electronic device 100 by communication of Bluetooth low energy. In this way, the communication between the electronic device 200 and the wearable device 100 can meet the medical regulations regarding electromagnetic interference (EMI) to avoid the invisible impact of electromagnetic waves on the human body and the problem of instrument failure due to electromagnetic interference. The electronic device 200 can determine a physiological status of the user in response to variations in the sampled body temperature signal BTS_S, the sampled audio signal AS_S, the sampled sensing capacitance value SCV_S and the sampled activity status ACT_S.

The electronic device 200 of this embodiment may be a device with a computing function such as a mobile phone, a tablet computer, a notebook computer or a desktop computer.

It is worth noting that, the wearable device 100 can sense the body temperature signal BTS of the user, the audio signal AS of the respiratory tract, the capacitance sensing signal SCV of the respiratory tract and the activity status ACT of the user to generate the sampled body temperature signal BTS_S, the sampled audio signal AS_S, the sampled sensing capacitance value SCV_S and the sampled activity status ACT_S. The electronic device 200 further determines the physiological status of the user in response to the variations in the sampled body temperature signal BTS_S, the sampled audio signal AS_S, the sampled sensing capacitance value SCV_S and the sampled activity status ACT_S. The user can use the physiological sensing system 10 at any place (e.g., at home or in a hospital). In this way, compared with collecting secretions from the throat or nasal cavity and chest X-ray examination, the physiological sensing system 10 can realize the long-term monitoring on the respiratory tract status and improve the convenience of detection.

Next, the implementation content for determining the physiological status of the user will be described as follows. In this embodiment, before the physiological sensing system 10 is used, the electronic device 200 generates an initial body temperature value and an initial capacitance value. The initial body temperature value is approximately equal to a surface body temperature in a healthy state. The initial body temperature value is, for example, 36° C. The initial capacitance value is approximately equal to the sensing capacitance value of the respiratory tract in the healthy state.

The electronic device 200 continuously records the sampled body temperature signal BTS_S. The electronic device 200 can provide an alert message when a body temperature value of the sampled body temperature signal BTS_S rises to be higher than the initial body temperature value by a preset value. In this embodiment, the reset value may be, for example, 2° C. or 5% of the initial body temperature value (i.e., 1.8° C.). Accordingly, the electronic device 200 can determine that a body temperature of the user is overly high when the body temperature value of the sampled body temperature signal BTS_S rises to be higher than the initial body temperature value by the preset value. The electronic device 200 can provide the alert message corresponding to the overly high body temperature. The initial body temperature value and the preset value of the invention are not limited to this embodiment.

In this embodiment, the electronic device 200 can continuously record the sampled sensing capacitance value SCV_S. The electronic device 200 can provide an alert message when the sampled sensing capacitance value SCV_S rises to be higher than the initial capacitance value by a preset value. The alveoli may be regarded as equivalent elements with resistors and capacitors. In general, healthy alveoli will contain air. Therefore, the equivalent capacitance value corresponding to healthy alveoli will be lower. When infiltration and/or edema start to occur in the lungs, multiple alveoli will accumulate liquid containing water, such as water, blood, and interstitial fluid. Therefore, the equivalent capacitance value corresponding to unhealthy alveoli will be higher. In this embodiment, the preset value may be, for example, 20% of the initial capacitance value. Accordingly, the electronic device 200 can determine that the lungs of the user are abnormal when the sampled sensing capacitance value SCV_S rises to be higher than 20% of the initial capacitance value. The electronic device 200 can provide the alert message corresponding to the abnormal lungs. The preset value of the invention is not limited to this embodiment.

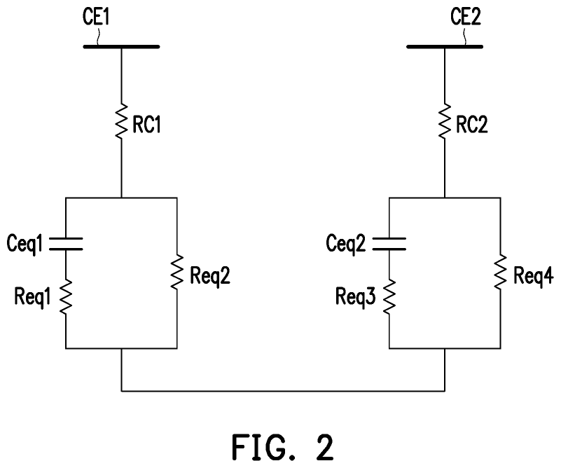

Next, the relationship between the sampled alveolar status and the sampled sensing capacitance value SCV_S will be described as follows. Referring to FIG. 1 and FIG. 2 together, FIG. 2 is an equivalent circuit diagram formed by a respiratory tract and capacitance detecting electrodes illustrated according to an embodiment of the invention. In this embodiment, the wearable device 100 further includes capacitance detecting electrodes CE1 and CE2. The capacitance detecting electrodes CE1 and CE2 are coupled to the capacitance sensor 113. The capacitance sensor 113 receives the capacitance sensing signal SCV through the capacitance detecting electrodes CE1 and CE2. In this embodiment, when the wearable device 100 is attached to the chest of the user, the capacitance detecting electrodes CE1 and CE2 may be in contact with the chest. Multiple alveoli corresponding to the capacitance detecting electrode CE1 may be equivalent to a first equivalent circuit. The first equivalent circuit includes an equivalent capacitor Ceq1 and equivalent resistors Req1 and Req2. Multiple alveoli corresponding to the capacitance detecting electrode CE2 may be equivalent to a second equivalent circuit. The second equivalent circuit includes an equivalent capacitor Ceq2 and equivalent resistors Req3 and Req4. In addition, a body tissue between the capacitance detecting electrode CE1 and the alveoli may be equivalent to an equivalent resistor RC1, and a body tissue between the capacitance detecting electrode CE2 and the alveoli may be equivalent to an equivalent resistor RC2. The capacitance detecting electrodes CE1 and CE2, the first equivalent circuit, the second equivalent circuit and the equivalent resistors RC1 and RC2 are serially coupled to each other. An equivalent capacitance value of the equivalent circuit diagram shown in FIG. 2 corresponds to an equivalent capacitance value formed by the equivalent capacitors Ceq1 and Ceq2 serially connected. Therefore, the sampled sensing capacitance value SCV_S corresponding to the equivalent capacitance value between the capacitance detecting electrodes CE1 and CE2 is related to Formula (1).

$$SCV\_S ∝ {ɛ}_{0} {ɛ}_{r} \frac{A}{D} \qquad Formula ( 1 )$$

In formula (1), co is a vacuum permittivity. The vacuum dielectric constant co is a fixed value. A is a contact area of the capacitance detecting electrodes CE1 and CE2. D is a sensing distance corresponding to FIG. 2. When the wearable device 110 is attached to the same position, values of A and D are unchanged. εr is a relative permittivity, and the relative permittivity εr changes according to the alveoli status. Therefore, based on Formula (1), the sampled sensing capacitance value SCV_S will be proportional to the relative permittivity εᵣ. The alveoli of healthy lung will contain air. The relative permittivity of air is 1, while the relative permittivity of water is 80. The relative permittivity of water is significantly higher than that of air. When the function of the lung is abnormal such that infiltration and/or edema occur, the alveoli of the lung will accumulate water, and the sampled sensing capacitance value SCV_S will rise. Accordingly, the electronic device 200 can determine that the lungs of the user are abnormal in response to rising of the sampled sensing capacitance value SCV_S. The number of the capacitance detecting electrodes of the invention may be multiple. The number of the capacitance detecting electrodes of the invention is not limited to this embodiment.

Referring back to the embodiment of FIG. 1, in this embodiment, the electronic device 200 can continuously record the sampled audio signal AS_S. The electronic device 200 provides an alert message when a frequency of the sampled audio signal AS_S rises to be a preset frequency. In general, a frequency of the breath sounding is approximately 100 Hz. When the secretions of the respiratory tract (e.g., upper respiratory tract and/or lower respiratory tract) increase, the gas of the respiratory tract will generate turbulence, so that the frequency of the respiratory tract sound is increased. In this embodiment, the preset frequency may be, for example, 1000 Hz. Accordingly, the electronic device 200 can determine that the respiratory tract of the user is abnormal when the frequency of the sampled audio signal AS_S rises to the preset frequency (i.e., 1000 Hz). The electronic device 200 can provide the alert message corresponding to the abnormal respiratory tract. The preset frequency of the invention is not limited to this embodiment.

The electronic device 200 can continuously record the sampled activity status ACT_S. The sampled activity status ACT_S can correspond to a mobility of the user. When the sampled activity status ACT_S indicates that the user has an insufficient mobility, the electronic device 200 provides an alert message corresponding to the insufficient mobility.

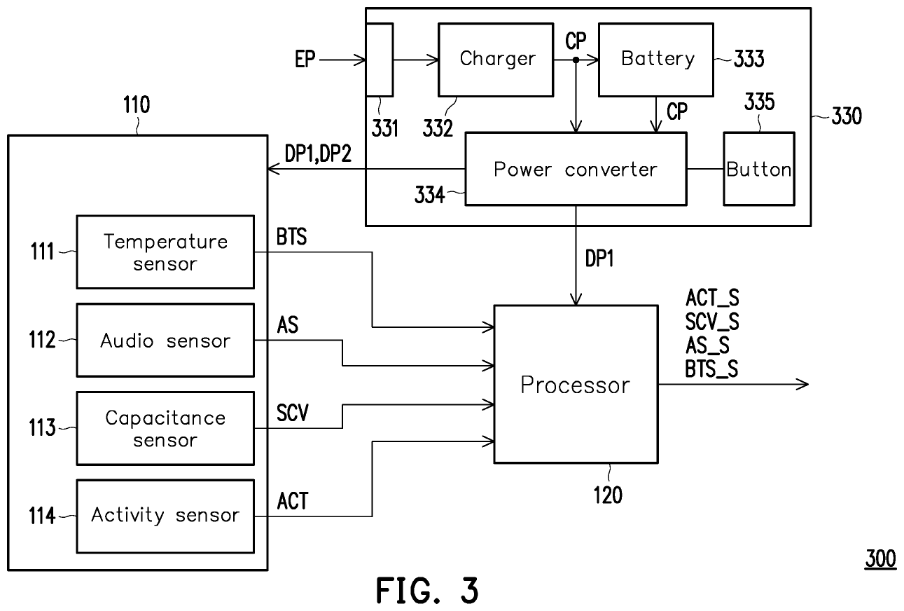

Referring to FIG. 3, FIG. 3 is a schematic diagram of a wearable device illustrated according to an embodiment of the invention. Unlike to the wearable device 100 shown by FIG. 1, a wearable device 300 of the present embodiment further includes a power module 330. The power module 330 can provide driving powers DP1 and DP2 for driving the sensing module 110 and the processor 120. In this embodiment, the power module 330 includes a power input port 331, a charger 332, a battery 333, a power converter 334 and a button 335. The power input port 331 receives an external power EP. The charger 332 is coupled to the power input port 331. The charger 332 receives the external power EP through the power input port 331 and converts the external power EP into a charging power CP. The battery 333 is coupled to the charger 332. The battery 333 stores the charging power CP. The power converter 334 is coupled to the charger 332 and the battery 333. The power converter 334 converts the charging power CP into the driving powers DP1 and DP2. The button 335 is coupled to the power converter 334. The button 335 is operated to control operations of the power converter 334. In this embodiment, the user may operate the button 335 to enable or disable the power converter 334.

For instance, when the external power EP is received by the power input port 331, the charger 332 is driven by the external power EP to provide the charging power to the battery 333 so as to charge the battery 333. When the power converter 334 is enabled, the power converter 334 converts the charging power CP into the driving powers DP1 and DP2. The power converter 334 drives the processor 120 by the driving power DP1, and drives the temperature sensor 111, the audio sensor 112, the capacitance sensor 113 and the activity sensor 114 by the driving powers DP1 and DP2.

When the power input port 331 does not receive the external power EP, the charger 332 cannot be driven. Accordingly, when the power converter 334 is enabled, the battery 333 provides the stored charging power CP to the power converter 334. The power converter 334 converts the charging power CP into the driving powers DP1 and DP2.

In certain embodiments, the processor 120 may also monitor a power consumption status of the wearable device 300 and provide information corresponding to the power consumption status to the electronic device 200. In this way, the user can learn of the power consumption status of the wearable device 300 through the electronic device 200. In certain embodiments, the processor 120 is further coupled to the battery 333. The processor 120 can learn of a power currently stored in the battery 333 and provide information corresponding to the power stored in the battery 333 to the electronic device 200. In this way, the user can learn of the power stored in the battery 333 through the electronic device 200.

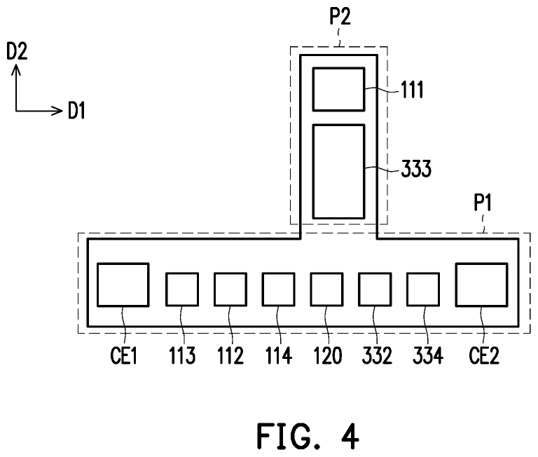

Referring to FIG. 3 and FIG. 4 together, FIG. 4 is a schematic diagram of a wearable device illustrated according to another embodiment of the invention. In this embodiment, the wearable device 300 is designed to include a first portion P1 and a second portion P2. The first portion P1 extends along a direction D1. The second portion P2 extends along a direction D2. The direction D1 is different from the direction D2. In this embodiment, the capacitance detecting electrodes CE1 and CE2 are disposed in the first portion P1. The temperature sensor 111 is disposed in the second portion P2 and away from the first portion P1. In other words, the temperature sensor 111 will be disposed away from the first portion P1 in the direction D2. In this embodiment, based on the first portion P1 and the second portion P2, a shape of the wearable device 300 may be an asymmetric “T” shape. In this embodiment, the shape of the wearable device 300 may be designed as a symmetric “T” shape. In certain embodiments, the shape of the wearable device 300 may be designed as an “L” shape. In this embodiment, the direction D1 is approximately perpendicular to the direction D2. In certain embodiments, the direction D1 is not perpendicular to the direction D2. The shape of the wearable device of the invention is not limited to this embodiment. In this embodiment, the entire wearable device 300 may be covered by a waterproof structure.

For example, in this embodiment, the audio sensor 112, the capacitance sensor 113, the activity sensor 114, the processor 120, the charger 332, and the power converter 334 may be disposed in the first portion P1. The battery 333 may be disposed in the second portion P2.

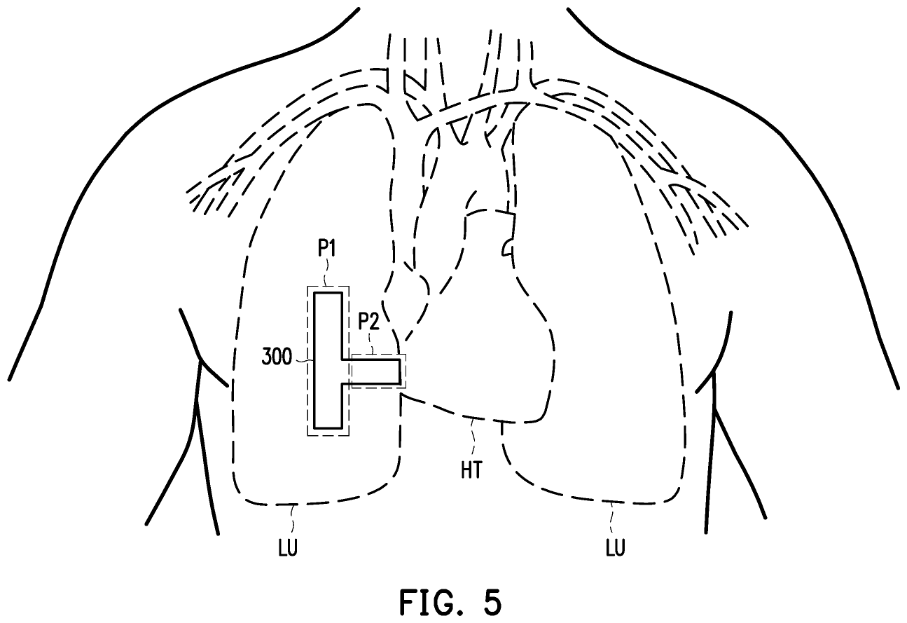

Referring to FIG. 4 and FIG. 5 together, FIG. 5 is a schematic diagram of a usage scenario of the wearable device illustrated according to an embodiment of the invention. In this embodiment, a lower half of lungs LU or below is a position where infiltration and/or edema are more likely to occur. In addition, a blood flow rate near a heart HT is faster. The temperature on the chest close to the position of the heart HT will be close to the core body temperature of the human body. The wearable device 300 may be attached to the chest and close to the lower half of the lungs LU or below. The second part P2 of the wearable device 300 will face toward the position of the heart HT. In this way, it is easier for the wearable device 300 to determine whether infiltration and/or edema occur in the lungs LU, and the body temperature signal BTS sensed can be close to the core temperature of the human body.

In summary, the physiological sensing system of the invention can sense the body temperature signal of the user, the audio signal of the respiratory tract, the capacitance sensing signal of the respiratory tract and the activity status of the user to generate the sampled body temperature signal, the sampled audio signal, the sampled sensing capacitance value and the sampled activity status. The invention can determine the physiological status of the user in response to variations in the sampled body temperature signal, the sampled audio signal, the sampled sensing capacitance value and the sampled activity status. In this way, the physiological sensing system can realize the long-term monitoring on the respiratory tract status and improve the convenience of detection.

Although the present disclosure has been described with reference to the above embodiments, it will be apparent to one of ordinary skill in the art that modifications to the described embodiments may be made without departing from the spirit of the disclosure. Accordingly, the scope of the disclosure will be defined by the attached claims and not by the above detailed descriptions.

1. A physiological sensing system, comprising:
a wearable device, comprising:
a sensing module, configured to sense a body temperature signal of a user, sense an audio signal of a respiratory tract of the user, sense a capacitance sensing signal of the respiratory tract, and sense an activity status of the user; and
a processor, coupled to the sensing module, and configured to sample the body temperature signal, the audio signal and the activity status to generate a sampled body temperature signal, a sampled audio signal and a sampled activity status and generate a sampled sensing capacitance value according to the capacitance sensing signal; and
an electronic device, configured to communicate with the wearable device to receive the sampled body temperature signal, the sampled audio signal, the sampled sensing capacitance value and the sampled activity status and determine a physiological status of the user in response to the sampled body temperature signal, the sampled audio signal, the sampled sensing capacitance value and the sampled activity status.

2. The physiological sensing system of claim 1, wherein the sensing module comprises:
a temperature sensor, configured to continuously sense the body temperature signal;
an audio sensor, configured to continuously sense the audio signal;
a capacitance sensor, configured to continuously sense the capacitance value; and
an activity sensor, configured to continuously sense the activity status.

3. The physiological sensing system of claim 2, wherein:
the wearable device further comprises:
a plurality of capacitance detecting electrodes, coupled to the capacitance sensor, the capacitance sensor receiving the capacitance sensing signal through the capacitance detecting electrodes.

4. The physiological sensing system of claim 3, wherein:
the wearable device is designed to include a first portion and a second portion,
the first portion extends along a first direction,
the second portion extends along a second direction different from the first direction,
the capacitance detecting electrodes are disposed in the first portion, and
the temperature sensor is disposed in the second portion and away from the first portion.

5. The physiological sensing system of claim 1, wherein the processor is further configured to:
filter the sampled audio signal to filter out signals outside a preset frequency range, and
filter the sampled sensing capacitance value to filter out noises.

6. The physiological sensing system of claim 1, wherein the processor is further configured to calculate, according to the capacitance sensing signal, the sampled sensing capacitance value corresponding to an equivalent capacitance value generated by an alveolar status in the respiratory tract.

7. The physiological sensing system of claim 6, wherein:
the electronic device is further configured to generate an initial body temperature value and an initial capacitance value,
the initial body temperature value is approximately equal to a body temperature in a healthy state, and
the initial capacitance value is approximately equal to the sensing capacitance value of the respiratory tract in the healthy state.

8. The physiological sensing system of claim 7, wherein the electronic device is further configured to:
record the sampled body temperature signal, and
provide an alert message when a body temperature value of the sampled body temperature signal rises to be higher than the initial body temperature value by a preset value.

9. The physiological sensing system of claim 7, wherein the electronic device is further configured to:
record the sampled sensing capacitance value, and
provide an alert message when the sampled sensing capacitance value rises to be higher than the initial capacitance value by a preset value.

10. The physiological sensing system of claim 1, wherein the electronic device is further configured to:
record the sampled audio signal, and
provide an alert message when a frequency of the sampled audio signal rises to be a preset frequency.

11. The physiological sensing system of claim 1, wherein the wearable device communicates with the electronic device by Bluetooth low energy.

12. The physiological sensing system of claim 1, wherein the wearable device further comprises:
a power module, coupled to the wearable device, and configured to provide at least one driving power for driving the sensing module and the processor.

13. The physiological sensing system of claim 12, wherein the power module comprises:
a power input port, configured to receive an external power;
a charger, coupled to the power input port, and configured to convert the external power into a charging power;
a battery, coupled to the charger, and configured to store the charging power;
a power converter, coupled to the charger and the battery, and configured to convert the charging power into the at least one driving power; and
a button, coupled to the power converter, and operated to control operations of the power converter.
